(19) United States
(12) Patent Application Publication
(10) Pub. No.: US 2025/0332097 A1
(43) Pub. Date: Oct. 30, 2025

(54) POROUS HYDROGEL MICROSPHERES AND PREPARATION METHODS THEREOF

(71) Applicant: SICHUAN CELL TISSUE BANK ENGINEERING TECHNOLOGY CO., LTD., Chengdu (CN)

(72) Inventors: Nan JIANG, Chengdu (CN); Guoliang YING, Chengdu (CN); Cui YE, Chengdu (CN); Xinyan LI, Chengdu (CN)

(73) Assignee: SICHUAN CELL TISSUE BANK ENGINEERING TECHNOLOGY CO., LTD., Chengdu (CN)

(21) Appl. No.: 19/026,269

(22) Filed: Jan. 16, 2025

(30) Foreign Application Priority Data
Apr. 28, 2024 (CN) 202410523653.1

(51) Int. Cl. A61K 9/06; A61K 35/28; A61K 35/33; A61K 47/06; A61K 47/34; A61K 47/42

(52) U.S. Cl. CPC A61K 9/06; A61K 35/28; A61K 35/33; A61K 47/06; A61K 47/34; A61K 47/42

(57) ABSTRACT

A porous hydrogel microsphere and the preparation method are provided thereof, the method comprising: mixing a gelation phase and a pore-forming phase at a temperature of 5-15° C. using a droplet microfluidic chip, and forming first droplets through a shear force; the gelation phase including gelatin methacryloyl, polyethylene glycol diacrylate, and a photoinitiator, and the pore-forming phase including polyethylene oxide and gelatin; then shearing a droplet stream formed by the first droplets through an oil phase to form second droplets; curing the second droplets by ultraviolet light irradiation to form a hydrogel microsphere, and removing the pore-forming phase from the hydrogel microsphere to obtain a porous hydrogel microsphere. The porous hydrogel microsphere with stable morphology can be prepared, and the porous structure of the porous hydrogel microsphere is conducive to cell proliferation and expansion.

### CROSS-REFERENCE TO RELATED APPLICATIONS

This application claims priority to Chinese Patent Application No. 202410523653.1, filed on Apr. 28, 2024, the contents of which are hereby incorporated by reference to its entirety.

### TECHNICAL FIELD

The present disclosure generally relates to the field of biomaterial technology, and in particular to a porous hydrogel microsphere and a preparation method thereof.

### BACKGROUND

Osteoarticular cartilage disease is a long-term chronic disease, which is one of the most common degenerative musculoskeletal diseases in the world. Due to the lack of blood vessels, nerves, endogenous repair cells, and growth factors in the degenerated or diseased joints, and the influence of various factors such as the depth, size, age, and location of the osteoarticular cartilage defect, the self-regeneration and renewal ability of the degenerated or diseased joints is greatly limited. Therefore, the treatment of osteoarticular cartilage disease remains a challenge.

Based on the challenging issue mentioned above, it is necessary to provide a biomaterial with good biocompatibility, which is capable of supporting cell growth and stretching and promoting cartilage regeneration, so as to effectively repair osteoarticular cartilage defects and improve the joint function of patients.

### SUMMARY

One or more embodiments of the present disclosure provide a method for preparing a porous hydrogel microsphere, comprising: mixing a gelation phase and a pore-forming phase at a temperature of 5-15° C. and forming first droplets through a shear force using a droplet microfluidic chip; wherein the gelation phase comprises gelatin methacryloyl, polyethylene glycol diacrylate, and photoinitiator; a concentration of gelatin methacryloyl in the gelation phase is within a range of 0.05-0.15 g/mL, a volume concentration of polyethylene glycol diacrylate is within a range of 0.5-3%, and a concentration of the photoinitiator is within a range of 0.001-0.005 g/mL; and the pore-forming phase comprises polyethylene oxide and gelatin, a concentration of polyethylene oxide in the pore-forming phase is within a range of 0.01-0.016 g/mL, and a concentration of gelatin is within a range of 0.05-0.1 g/mL; shearing a droplet stream formed by the first droplets through an oil phase to form second droplets; curing the second droplets under ultraviolet light to form a hydrogel microsphere; and removing the pore-forming phase from the hydrogel microsphere to obtain the porous hydrogel microsphere; wherein the droplet microfluidic chip includes: a first liquid injection hole, configured to introduce the gelation phase, a second liquid injection hole, configured to introduce the pore-forming phase, a third liquid injection hole, configured to introduce the oil phase, a first flow channel connected to the first liquid injection hole, a second flow channel connected to the second liquid injection hole, and a third flow channel connected to the third liquid injection hole, the second flow channel being provided with a plurality of branch flow channels spaced apart from each other, each of the branch flow channels being arranged perpendicular to the first flow channel to form a T-shaped shear, and the third flow channel crossing the first flow channel in a cross shape.

One or more embodiments of the present disclosure provide a porous hydrogel microsphere prepared by the method for preparing a porous hydrogel microsphere as described above.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are non-limiting exemplary embodiments, in which like reference numerals represent similar structures throughout the several views of the drawings, wherein:

FIG. 1 is an exemplary flowchart of a method for preparing a porous hydrogel microsphere according to some embodiments of the present disclosure;

FIG. 2 is an image of porous hydrogel microspheres using an optical microscope according to some embodiments of the present disclosure;

FIG. 3 is an image of porous hydrogel microspheres using a fluorescence microscope according to some embodiments of the present disclosure;

FIG. 4 is an image of porous hydrogel microspheres using a fluorescence microscope according to some embodiments of the present disclosure;

FIG. 5 is an image illustrating biocompatibility of porous hydrogel microspheres according to some embodiments of the present disclosure;

FIG. 6 is an image illustrating expansion and proliferation of bone marrow mesenchymal stem cells in porous hydrogel microspheres after one day according to some embodiments of the present disclosure;

FIG. 7 is an image illustrating expansion and proliferation of bone marrow mesenchymal stem cells in porous hydrogel microspheres after 7 days according to some embodiments of the present disclosure;

FIG. 8 is a two-dimensional morphological diagram of bone marrow mesenchymal stem cells in porous hydrogel microspheres according to some embodiments of the present disclosure;

FIG. 9 is a three-dimensional morphological diagram of bone marrow mesenchymal stem cells in porous hydrogel microspheres according to some embodiments of the present disclosure;

FIG. 10 is a fluorescence microscopy image of a gelation phase and a pore-forming phase mixed at temperatures of 5° C., 15° C., and 25° C. and maintained after 120 s according to some embodiments of the present disclosure;

FIG. 11 is a diagram illustrating the phase separation of a pore-forming phase without loaded cells at temperatures of 4° C., 8° C., 16° C., 24° C., and 37° C. according to some embodiments of the present disclosure;

FIG. 12 is an exemplary schematic diagram of a droplet microfluidic chip according to some embodiments of the present disclosure;

FIG. 13 is a diagram illustrating the cross arrangement of a first flow channel and a third flow channel of a droplet microfluidic chip according to some embodiments of the present disclosure.

In the drawings: 11—first liquid injection hole; 12—second liquid injection hole; 13—third liquid injection hole; 14—first flow channel; 15—second flow channel; 151—branch flow channel; 16—third flow channel; 17—fourth flow channel; and 18—fifth flow channel.

### DETAILED DESCRIPTION

As disclosed herein and in the claims, unless otherwise indicated by the context, the words “one”, “a”, “a kind of”, and/or “the” are not limited to the singular form and may also encompass the plural. Generally, the terms “including” and “comprising” indicate the inclusion of explicitly identified steps and elements. The listed steps and elements are not exclusive, and the method or apparatus may include other steps or elements.

As used herein, the term “biomaterial” refers to materials that can interact with biological systems and are generally used in the medical or biomedical fields to replace, repair, enhance, or support the function of tissues or organs. Biomaterials may be natural, synthetic, or a combination of both, and have properties compatible with organisms, such as good biocompatibility, appropriate mechanical properties, and low toxicity to biological systems.

As used herein, the terms “administering”, “introducing”, “delivering”, “placing” and “transplanting” are used interchangeably and refer to placing the porous hydrogel microsphere in the embodiments of the present disclosure into the subject's body by a method or route that partially or completely directs the cells and/or porous hydrogel microspheres to the desired location (e.g., a target location). The cells and/or porous hydrogel microsphere may be administered by any suitable route that is capable of delivering the cells and/or porous hydrogel microsphere to the target location in the subject's body, where the therapeutic ability of the cells and/or porous hydrogel microsphere is at least partially retained. For example, exemplary administration methods may include intravenous administration.

As used herein, the term “treatment” includes introducing or applying porous hydrogel microspheres prepared according to the embodiments of the present disclosure into the subject's body by any means to reduce or alleviate at least one adverse effect or symptom of a disease or defect, such as cartilage defect.

As used herein, the term “effective amount” refers to an amount of porous hydrogel microspheres sufficient to achieve a beneficial or desired result. An effective amount may be administered through one or more administrations, applications, or dosages and not limited to a particular formulation or administration route.

As used herein, the term “host”, “patient” or “subject” refers to an organism that will be treated with the formulations and/or methods of the embodiments of the present disclosure, or an organism that is subjected to various tests provided by the present technology. The term “subject” includes animals, preferably mammals, including humans. In some embodiments, the subject is a primate. In some embodiments, the subject is a human.

Gelatin methacryloyl (GelMA) hydrogel is a high molecular cross-linked hydrophilic polymer with high water content, high biocompatibility, and extracellular matrix (ECM) simulation ability, making it an ideal candidate biomaterial. Porous hydrogel microspheres (HMs) can not only carry cells, but also better support cell growth and cell interactions due to the high surface to volume ratio (i.e., specific surface area), which can accurately simulate ECM. In addition, GelMA has ideal mechanical properties and a multilayer structure with a biological gradient distribution, which can achieve regeneration of joint lesions and is an excellent material for treating cartilage defects.

Based on this, the embodiments of the present disclosure provide a porous hydrogel microsphere and a preparation method and application thereof, which can form porous GelMA hydrogel microspheres with stable morphology, the porous structure of which is conducive to cell proliferation and expansion.

In the first aspect, the embodiments of the present disclosure provide a method for preparing porous hydrogel microspheres.

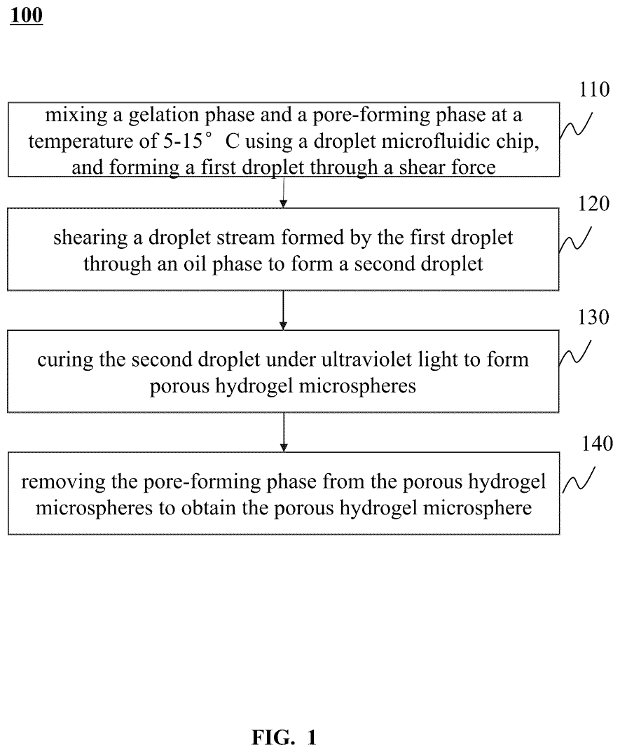

FIG. 1 is an exemplary flowchart of a method for preparing porous hydrogel microspheres according to some embodiments of the present disclosure. As shown in FIG. 1, the process 100 includes the following operations.

Operation 110, mixing a gelation phase and a pore-forming phase at a temperature of 5-15° C. and forming first droplets through a shear force using a droplet microfluidic chip.

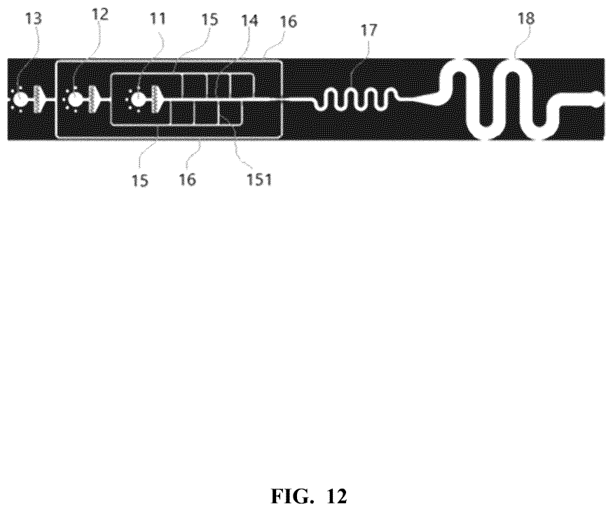

The droplet microfluidic chip is an experimental platform based on microfluidic technology. By controlling the flow of liquid in tiny channels, micron-sized droplets may be generated, manipulated, and analyzed. The details regarding the droplet microfluidic chip may be found in the following description (for example, FIG. 12 and its related description).

The gelation phase refers to a raw material or solution that is converted into a gel-like substance under appropriate conditions by cross-linking or other means. For example, the gelation phase may include a material that is converted into a gel under conditions such as a cross-linking reaction, temperature change, pH change, or illumination. Exemplary gelation phase may include gelatin, sodium alginate, polyvinyl alcohol, carrageenan, and polyurethane.

In some embodiments, the gelation phase may include gelatin methacryloyl and polyethylene glycol diacrylate.

In some embodiments, the gelation phase may further include a photoinitiator. As a part of the gelation phase, the photoinitiator may initiate a polymerization reaction or a cross-linking reaction under the irradiation of ultraviolet light or visible light to promote the formation of a gel. Exemplary photoinitiator may include phenyl benzophenone and benzoic acid esters.

In some embodiments, the photoinitiator is lithium phenyl (2,4,6-trimethylbenzoyl) phosphinate (LAP).

In some embodiments, a concentration of at least one component in the gelation phase may be adjusted to achieve a best gelation effect. The gelation effect of the hydrogel may be characterized by the expansion and proliferation of cells in the prepared hydrogel.

In some embodiments, a mass concentration of gelatin methacryloyl in the gelation phase is within a range of 0.05-0.15 g/mL, a volume concentration of polyethylene glycol diacrylate in the gelation phase is within a range of 0.5-3%, a mass concentration of the photoinitiator in the gelation phase is within a range of 0.001-0.005 g/mL.

In some embodiments, the mass concentration of gelatin methacryloyl in the gelation phase may be any one of 0.05 g/mL, 0.08 g/mL, 0.10 g/mL, 0.12 g/mL, and 0.15 g/mL, or a value between any two of them; the volume concentration of polyethylene glycol diacrylate in the gelation phase may be any one of 0.5%, 1.0%, 1.3%, 1.6%, 2.0%, 2.5%, and 3%, or a value between any two of them; and the mass concentration of LAP in the gelation phase may be 0.001 g/mL, 0.003 g/mL, or 0.005 g/mL.

The pore-forming phase refers to a material that forms a pore structure through physical or chemical treatment after forming the gel. The pore-forming phase may include polymer microspheres such as polylactic acid, polystyrene, polymethyl methacrylate (PMMA), etc. polyurethane, polyvinyl alcohol-pentene (PVA-PEG) composite materials, and porous silica gel.

In some embodiments, the pore-forming phase may include polyethylene oxide and gelatin.

In some embodiments, a mass concentration of polyethylene oxide in the pore-forming phase is within a range of 0.01-0.016 g/mL and a mass concentration of gelatin in the pore-forming phase is within a range of 0.05-0.1 g/mL.

In some embodiments, the mass concentration of polyethylene oxide in the pore-forming phase may be any one of 0.01 g/mL, 0.012 g/mL, 0.014 g/mL, and 0.016 g/mL, or a value between any two of them; and the mass concentration of gelatin in the pore-forming phase may be any one of 0.05 g/mL, 0.07 g/mL, 0.08 g/mL, and 0.1 g/mL, or a value between any two of them.

In some embodiments, cells are loaded in the pore-forming phase. Further details regarding the preparation of cell-loaded porous hydrogels may be found in the following description.

In some embodiments, the temperature of mixing the gelation phase and the pore-forming phase may be controlled to any one of 5° C., 6°° C., 7° C., 8° C., 9° C., 10° C., 11° C., 12° C., 13° C., 14° C., and 15° C., or a value between any two of them. In some embodiments, the temperature control is carried out by cooling, specifically, cooling by using a semiconductor chilling plate. It will be understood that in other embodiments, cooling may be achieved by alternative methods such as water cooling, as long as the desired cooling effect can be achieved.

In some embodiments, the mixing temperature of the gelation phase and the pore-forming phase may be regulated by a temperature control component.

The temperature control component refers to a component that regulates the temperature. For example, the temperature control component may include a heating component and a cooling component.

In some embodiments, the droplet microfluidic chip may communicate with the processor and the temperature control component. For example, the processor is connected to the droplet microfluidic chip through a data bus or a dedicated communication interface (e.g., I2C, SPI, UART, etc.). A temperature sensor in the droplet microfluidic chip may send temperature data to the processor. After receiving the temperature data, the processor may generate a control instruction based on the temperature data and send the control instruction to the temperature control component (heating or cooling component) to instruct the temperature control component to increase or decrease the temperature, to control the fluid operation, reaction conditions, or other experimental parameters in the droplet microfluidic chip.

In some embodiments, the processor may determine the mixing temperature of the gelation phase and the pore-forming phase in the droplet microfluidic chip by querying a first preset table based on a pore size requirement.

The pore size requirement refers to a pore size of the porous hydrogel microspheres to be obtained. In some embodiments, the pore size requirement may be directly input by the user based on historical pore size data.

The first preset table may include pore size requirement and the mixing temperature of the gelation phase and the pore-forming phase corresponding to the pore size requirement. In some embodiments, the first preset table may be constructed based on historical data.

In some embodiments, the processor may filter the historical data based on pore size uniformity to obtain the first preset table. In some embodiments, the processor may use the historical pore size data with high pore size uniformity and a mixing temperature of the gelation phase and the pore-forming phase corresponding to the historical pore size data as data in the first preset table.

Pore size uniformity may be characterized in various ways. For example, the pore size uniformity may be measured using the variance of the pore sizes of the obtained hydrogel microspheres. The smaller the variance, the higher the pore size uniformity.

In some embodiments of the present disclosure, when the temperature is too high, the pore-forming phase of the small droplets distributed uniformly in the gelation phase accelerates mutual diffusion and fusion to form large droplets of polyethylene oxide, so as to cause the uniformly distributed and interconnected pores to become a single large pore, which is not conducive to forming a porous structure with a uniform size and a stable structure. Precise regulation by the temperature control component can effectively ensure the uniformity of the pore size.

In some embodiments, the processor may also determine a plurality of groups of candidate mixing temperature, determine the pore size uniformity based on the candidate mixing temperature and the pore size requirement through a pore size uniformity prediction model, and determine the mixing temperature of the gelation phase and the pore-forming phase based on the pore size uniformity.

The candidate mixing temperature refers to a plurality of groups of mixing temperature within the range of 5 to 15° C. randomly generated by the processor.

The pore size uniformity prediction model refers to a model configured to determine the pore size uniformity. In some embodiments, the pore size uniformity prediction model may be a machine learning model such as a convolutional neural network (CNN), a recurrent neural network (RNN), etc.

In some embodiments, an input to the pore size uniformity prediction model may include the candidate mixing temperature and the pore size requirement. The output of the pore size uniformity prediction model may include the pore size uniformity.

In some embodiments, the processor may train the pore size uniformity prediction model based on a plurality of sets of first training samples with first training labels.

In some embodiments, a set of first training samples may include a sample candidate mixing temperature and a sample pore size requirement. The first training samples may be acquired based on historical data, and the first training labels may be pore size uniformities corresponding to the first training samples. The first training labels may be drawn and labeled by a processor and/or manually based on historical data.

In some embodiments, the processor may input the first training samples into an initial pore size uniformity prediction model, construct a first loss function based on the pore size uniformities output by the initial pore size uniformity prediction model and the first training labels, update parameters of the initial pore size uniformity prediction model based on the first loss function, and when a first preset condition is met, completing the training of the initial pore size uniformity prediction model to obtain a trained pore size uniformity prediction model. The first preset condition may be that the first loss function converges, the number of iterations reaches a preset threshold, etc.

In some embodiments, the processor may select the candidate mixing temperature corresponding to the highest pore size uniformity as the mixing temperature of the gelation phase and the pore-forming phase.

In some embodiments, the droplet microfluidic chip includes a plurality of liquid injection holes configured to inject the gelation phase and the pore-forming phase, and the input of the pore size uniformity prediction model may also include the injection temperature of at least one injection hole.

In some embodiments of the present disclosure, the pore size uniformity prediction model processes the pore size requirement and candidate mixing temperature to predict the pore size uniformities corresponding to a plurality of candidate mixing temperature and select the candidate mixing temperature corresponding to the highest pore size uniformity as the final mixing temperature of the gelation phase and the pore-forming phase, which can achieve automated optimization for the preparation process of the porous hydrogel microsphere, reduce manual intervention, and improve the convenience and efficiency of operation. In addition, the embodiments of the present disclosure input the injection temperature into the pore size uniformity prediction model, fully considering the impact of the injection temperature on the pore size uniformity, which can make the pore size uniformity output by the pore size uniformity prediction model more consistent with the results of actual operations.

According to the method for preparing porous hydrogel microspheres of the embodiments of the present disclosure, the gelation phase of the hydrogel and the pore-forming phase are mixed at the temperature of 5-15° C. and the first droplets are formed through a shear force. The temperature is controlled at a range of 5-15° C., which can make the gelation phase of the hydrogel and the pore-forming phase in a stably separated state. Although the gelation phase and the pore-forming phase are mixed, they are not in a fused state. In the study, it is found that temperature has a very important influence on the stability of a two-phase emulsion containing both the gelation phase of the hydrogel and the pore-forming phase. By controlling the mixing temperature of the gelation phase and the pore-forming phase, the prepared hydrogel microspheres can have a porous structure with a uniform size and a stable structure.

Operation 120, shearing a droplet stream formed by the first droplets through an oil phase to form second droplets.

The oil phase refers to a fluid medium that wraps the droplets externally during the droplet generation process. The main function of oil phase is to provide shear force to form and stabilize the droplets.

In some embodiments, the oil phase may include dimethylsilicone and a surfactant, and the surfactant may include decamethylcyclopentasiloxane and trimethylsiloxy silicate.

In some embodiments, the oil phase may further include a hydrofluoroether and a nonionic fluorocarbon surfactant, and a volume of the nonionic fluorocarbon surfactant may be 1-2% of a volume of the hydrofluoroether.

Operation 130, curing the second droplets under ultraviolet light to form hydrogel microspheres.

Ultraviolet light irradiation may activate the photoinitiator in the hydrogel to start the polymerization reaction, causing that covalent bonds are formed between small molecules in the hydrogel, thereby solidifying the droplets.

The rate and extent of the curing reaction may be precisely controlled based on the intensity and duration of ultraviolet light irradiation and the type of photoinitiator. Short irradiation time may result in incomplete curing of the hydrogel microsphere. Reasonable irradiation time may ensure that the droplets are completely cured to form uniform and stable hydrogel microspheres. Control of the intensity and duration of irradiation can affect the size, hardness, and internal structure of the hydrogel microspheres.

In some embodiments, the processor may determine a curing time of the second droplets based on at least one of current test results of second droplets.

The test results may include pore size uniformity and a content of pore-forming phase. In some embodiments, the pore size uniformity and pore-forming phase may be obtained by microscopic imaging or other methods. For example, the pore size uniformity may also be determined based on a pore size uniformity prediction model. Further details regarding the pore size uniformity prediction model may be found in the above relevant descriptions.

In some embodiments, when the difference between the pore size uniformity and the pore diameter uniformity threshold, or the content of pore-forming phase is smaller, the curing time under ultraviolet light is closer to the standard curing time.

The standard curing time refers to an ideal irradiation time of the ultraviolet light on the second droplets, which may be used to ensure that the second droplets are completely cured.

The pore size uniformity threshold refers to a standard value at which the pore size distribution uniformity reaches the preset requirement during the droplet curing process. When the difference between the pore size uniformity and the pore size uniformity threshold is small, it means that the pore size distribution is close to uniform, the curing process is more precise, and the droplet morphology is more stable. The pore size uniformity threshold may be preset according to an actual need or a specific application. In some embodiments of the present disclosure, when the irradiation time of ultraviolet light is not enough, the structural strength of the gelation phase is insufficient, and the micropores may be destroyed, resulting in changes or uniformness in pore size. If the current test results show that the pore size uniformity is not enough, the pore size uniformity may be balanced by adjusting the curing time.

In some embodiments, the droplet microfluidic chip may be placed in a rotating device, and the rotating device drives the droplet microfluidic chip to rotate at a certain rotation speed.

In some embodiments, the curing time of the second droplets may also be related to the rotation speed of the rotating device. For example, when the rotation speed is approximately close to a preset rotation speed, the curing time under the ultraviolet light is approximately close to a standard curing time. The preset rotation speed may be set by a technician or set based on experience and experimental results.

In some embodiments of the present disclosure, when the rotation speed is closer to the preset rotation speed, the pore size uniformity is better. At this time, the standard curing time should be maintained to improve the pore size uniformity. If the rotation speed deviates from the preset rotation speed at this time, it means that the pore size uniformity may be poor. By appropriately adjusting the curing time, the pore size uniformity may be balanced.

Operation 140, removing the pore-forming phase from the hydrogel microspheres to obtain the porous hydrogel microspheres.

The pore-forming phase may be removed in a variety of ways. For example, the hydrogel microsphere may be immersed in a suitable solvent to selectively dissolve the pore-forming phase. The pore-forming phase may be removed through washing and solvent exchange processes. Fluids such as supercritical carbon dioxide may also be used to be introduced into the pores of the hydrogel microsphere to remove the pore-forming phase so as to form a porous structure. The hydrogel microsphere may also be heated to an appropriate temperature to volatilize or degrade the pore-forming phase to remove the pore-forming phase. The present disclosure does not impose any limitations on the method for removing the pore-forming phase.

In some embodiments, the pore-forming phase is loaded with cells. In some embodiments, the cells may include bone marrow mesenchymal stem cells, fibroblasts, and adipose-derived mesenchymal stem cells.

By loading cells into the pore-forming phase and embedding the cells in porous hydrogel microspheres, it facilitates the simulation of the mechanical environment of cartilage matrix and growth and infiltration of cells. If the cells are loaded and co-cultured with the cells after the porous hydrogel microspheres are formed, most of the cells only adhere to the surface of the hydrogel microspheres and cannot effectively grow into the interior of the hydrogel microspheres.

Exemplarily, the operation of loading cells in the pore-forming phase may include: digesting and centrifuging the cultured cells, removing the supernatant, and then adding a certain number of cells (e.g., at a concentration of 10⁷ cells/mL) to the pore-forming phase and mixing; collecting the second droplets in a culture bottle containing culture medium, and irradiating second droplets under 15 W ultraviolet light for above 15 s to cure the second droplets to form hydrogel microspheres. Under the irradiation of 15 W ultraviolet light, the cell viability is not significantly damaged; when the hydrogel microspheres enter into the culture medium, gentle shaking allows the pore-forming phase to dissolve into the culture medium; the cured and crosslinked hydrogel microspheres and the cells adhered to its pore structure are then cultured in an incubator, culture medium is changed the next day, and the pore-forming phase and residual oil phase may be washed away.

In some embodiments, when cells are loaded in the pore-forming phase, the oil phase may include hydrofluoroether (a type of HFE-7500) and a nonionic fluorocarbon surfactant. When cells are loaded in the pore-forming phase, it is difficult to demulsify and collect the hydrogel microspheres for embedding cells generated by shearing of dimethylsilicone, while hydrofluoroether is volatile and has good biocompatibility. When embedding cells, the outlet of the microfluidic device may be directly connected to the culture medium to collect the hydrogel microspheres, and then the hydrofluoroether is volatilized. If the hydrofluoroether is not completely volatilized, it may also be removed when changing the fluid for the cells.

It should be noted that the droplet microfluidic chip of the embodiment of the present disclosure only needs to be able to achieve the above-mentioned function of forming the first droplets and the second droplets, and the specific structure is not limited.

In some embodiments, the droplet microfluidic chip may include: a first liquid injection hole configured to introduce a gelation phase, the first liquid injection hole being connected to a first flow channel; a second liquid injection hole configured to introduce a pore-forming phase, the second liquid injection hole being connected to a second flow channel; and a third liquid injection hole configured to introduce an oil phase, the third liquid injection hole being connected to a third flow channel.

In some embodiments, one or more branch flow channels may be spaced and disposed between the second flow channel and the first flow channel.

In some embodiments, each branch flow channel is connected to the first flow channel and the second flow channel and is perpendicular arranged to form a T-shaped shear, and the third flow channel is connected to the first flow channel and is arranged in a cross shape.

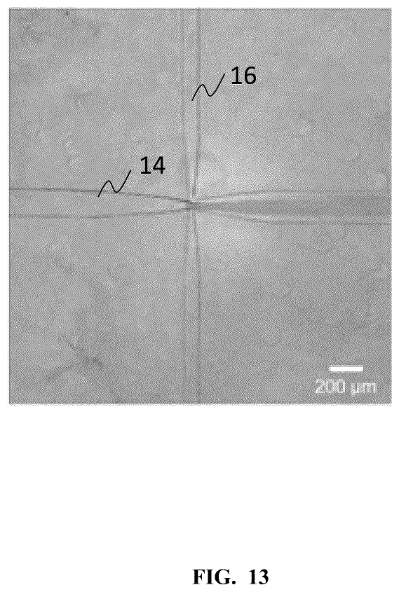

In some embodiments, the droplet microfluidic chip (refer to FIG. 12) includes a first liquid injection hole 11 for introducing a gelation phase, a second liquid injection hole 12 for introducing a pore-forming phase, a third liquid injection hole 13 for introducing an oil phase, a first flow channel 14 connected to the first liquid injection hole 11, a second flow channel 15 connected to the second liquid injection hole 12, and a third flow channel 16 connected to the third liquid injection hole 13. The second flow channel 15 is provided with a plurality of branch flow channels 151 arranged at intervals, each branch flow channel 151 is arranged perpendicular to the first flow channel 14 to form a T-shaped shear, and the third flow channel 16 is arranged with the first flow channel 14 in a cross shape (refer to FIG. 13).

The gelation phase is introduced into the first flow channel 14 from the first liquid injection hole 11, and the pore-forming phase is introduced into the second flow channel 15 from the second liquid injection hole. The second flow channel 15 has a plurality of branch flow channels 151, and each of the branch flow channels 151 is arranged perpendicular to the first flow channel 14 to form a T-shaped shear, so that the size and spatial distribution of the formed pores are slightly more uniform.

In some embodiments, the second liquid injection hole 12 is arranged in a shape of right-angled trapezoid, which is more conducive to fluid shearing.

In some embodiments, the first liquid injection hole 11, the second liquid injection hole 12, and the third liquid injection hole 13 are each provided with at least two rows of square holes, which can facilitate the discharge of bubbles, prevent larger impurities from entering, and make the liquid flow more stable.

In some embodiments, the droplet microfluidic chip is further provided with a fourth flow channel 17 and a fifth flow channel 18, the fourth flow channel 17 is connected to the outlet of the second droplets, the fifth flow channel 18 is connected to the fourth flow channel 17, the fifth flow channel 18 and the fourth flow channel 17 are both extended in an s-shape, the bending length of the fifth flow channel 18 is longer than the bending length of the fourth flow channel 17, and the inner diameter of the fifth flow channel 18 is larger than the inner diameter of the fourth flow channel 17. The s-shaped extension of the fourth flow channel 17 can be beneficial to the mixing of the droplets (making the pore-forming phase uniformly distributed) and the stable morphology (spherical), and the fifth flow channel 18 is beneficial to slowing down the flow rate for easy observation under the microscope.

In some embodiments, the liquid injection operation of different liquid injection holes of the droplet microfluidic chip may be regulated by an automatic liquid injection device, and the liquid injection order of the injection holes may be preset by the user.

In some embodiments, the droplet microfluidic chip may be disposed in a rotating device, and the rotating device drives the droplet microfluidic chip to rotate at a certain rotation speed.

In some embodiments of the present disclosure, the uniformity of the formed pore size can be improved by rotating the droplet microfluidic chip at a rotation speed through a rotating device.

In some embodiments, the processor may also determine the rotation speed of the rotating device based on the candidate mixing temperature of the gelation phase and the pore-forming phase, the pore size requirement, and the candidate rotation speed by using the pore size uniformity prediction model.

The candidate rotation speed refers to a plurality of groups of rotation speeds randomly generated by the processor. In some embodiments, the candidate rotation speed may also be determined based on the most frequently used rotation speed in historical data. The higher the pore size uniformity requirement, the more groups of candidate rotation speeds generated by the processor.

Further details regarding the pore size uniformity prediction model and its training process are provided in the above relevant descriptions.

In some embodiments, the processor may select a candidate rotation speed corresponding to the highest pore size uniformity output by the pore size uniformity prediction model as the actual rotation speed.

In some embodiments of the present disclosure, by controlling the mixing temperature of the gelation phase and the pore-forming phase and rotation speed, the properties of the prepared hydrogel microspheres can be improved, making the prepared hydrogel microspheres more suitable for supporting cell growth and expansion and promoting cartilage regeneration.

In a second aspect, the present disclosure provides a porous hydrogel microsphere, which is prepared by the above preparation method for the porous hydrogel microsphere.

It is verified that the porous hydrogel microspheres of the embodiments of the present disclosure have a porous structure that is conducive to cell proliferation and expansion, which may be used to treat cartilage defects.

In a third aspect, the present disclosure provides an application of the above porous hydrogel microspheres in the preparation of cartilage repair materials and osteogenic materials.

In a fourth aspect, the present disclosure provides a method for repairing cartilage defects, which may include: delivering an effective amount of porous hydrogel microspheres to the cartilage defect area in the subject by injection or local administration. The porous hydrogel microspheres provide support for cell growth and promote the repair of cartilage tissue through their porous structure. In some embodiments, the size and shape of the porous hydrogel microspheres are precisely controlled by adjusting the porosity and mechanical properties to precisely control the distribution and effect of the porous hydrogel microspheres in the cartilage defect area.

The treatment method can reduce or alleviate the pain or loss of function caused by cartilage defects and restore the normal function of the joint.

The following detailed description of the embodiments of the present disclosure is provided in conjunction with specific examples. However, those skilled in the art will appreciate that the embodiments are for illustrative purposes only and should not be construed as limiting the scope of the disclosure. For conditions not specifically mentioned in the embodiments, conventional conditions or those recommended by the manufacturer should be followed. Reagents or instruments not specified with a manufacturer are conventional products that may be obtained commercially.

### EXAMPLES

### Example 1

Example 1 of the present disclosure provided a method for preparing porous hydrogel microspheres, comprising the following operations.
(1) A droplet microfluidic chip was used to mix the gelation phase and the pore-forming phase at a temperature of 15° C. and form first droplets through shearing; the mass concentration of gelatin methacryloyl in the gelation phase was 0.1 g/mL, and the gelatin methacryloyl was dissolved in phosphate-buffered saline (PBS). The volume concentration of polyethylene glycol diacrylate in the gelation phase was 1%, and the mass concentration of lithium phenyl (2,4,6-trimethylbenzoyl) phosphinate (LAP photoinitiator) was 0.005 g/mL. The mass concentration of polyethylene oxide in the pore-forming phase was 0.016 g/mL, and the polyethylene oxide was dissolved in PBS. The mass concentration of gelatin in the pore-forming phase was 0.084 g/mL, and gelatin was dissolved in PBS. The pore-forming phase was loaded with bone marrow mesenchymal stem at a concentration of 2×10⁷ cells/mL.
(2) A droplet stream formed by the first droplets was sheared through an oil phase to form second droplets; and the oil phase included a hydrofluoroether and a non-ionic fluorocarbon surfactant, and the volume of the non-ionic fluorocarbon surfactant was 2% of the volume of the hydrofluoroether.
(3) The second droplets were collected in a culture bottle (with culture medium) and irradiated under 15 W ultraviolet light for about 15 s, so that the second droplets were cured to form hydrogel microspheres. When the hydrogel microspheres entered the culture medium, the hydrogel microspheres were slightly shaken to dissolve the pore-forming phase in the culture medium. The culture medium was changed the next day, and the pore-forming phase and residual oil phase were washed away during the changing the culture medium, thereby obtaining porous hydrogel microspheres.

### Comparative Example 1

Comparative Example 1 of the present disclosure provided a method for preparing porous hydrogel microspheres. Compared with Example 1, the only difference in the preparation process of the comparative Example 1 was that in step (1), the temperature was controlled at 25° C.

### Test Example 1

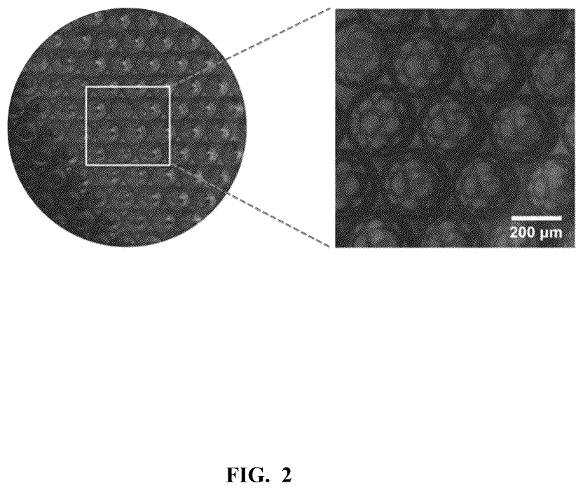

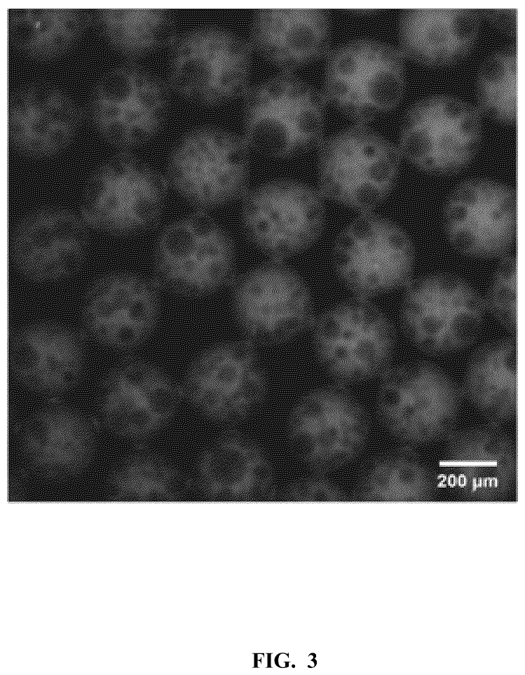

The porous hydrogel microspheres prepared in Example 1 were observed under both an optical microscope and a fluorescence microscope, and the results are illustrated in FIG. 2 and FIG. 3, respectively.

As shown in FIG. 2 and FIG. 3, the porous hydrogel microspheres prepared in Example 1 have both microscopic pores associated with the chemical structure and macroscopic pores formed after the removal of the pore-forming phase, which presents a spatial and three-dimensional hierarchical pore structure in the microspheres.

### Test Example 2

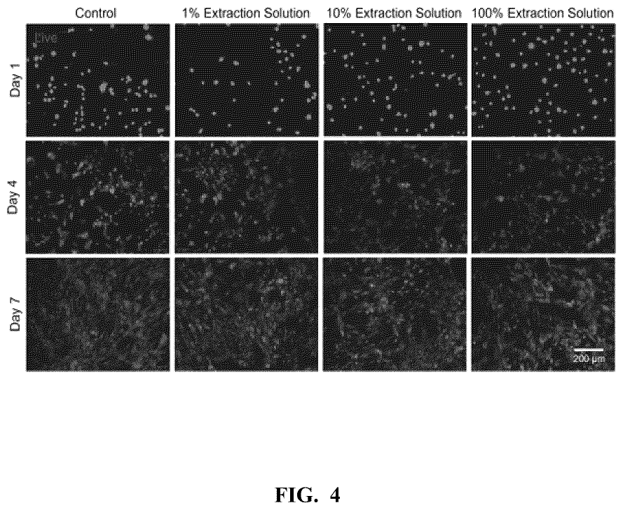

The porous hydrogel microspheres prepared in Example 1 were tested for biocompatibility, and the results are shown in FIG. 4. The test procedure was as follows.
1) Preparation of extraction solution

The sterile porous hydrogel microspheres were added to the culture medium at a concentration of 0.1 g/mL and extracted in a sealed glass container at 37° C. for 24 h, the supernatant was then aspirated and filtered to obtain the extraction solution with a concentration of 1 g/mL. During the test, the extraction solution was diluted with cell culture medium to the required concentration.
2) Cell preparation

The cultured fibroblasts were counted to a cell density of 1×10⁶ cells, the required volume of cell suspension was calculated, and the cells were resuspended in culture medium.
3) Addition of extraction solution

The resuspended cells were added to the diluted extraction solutions at different concentrations. 200 μL of the extraction solution was added to each well, with three rows of 96-well plates used for each dilution and two rows for blank control cell. The 96-well plates were then placed in an incubator at 37° C. for culture.
4) Cell staining

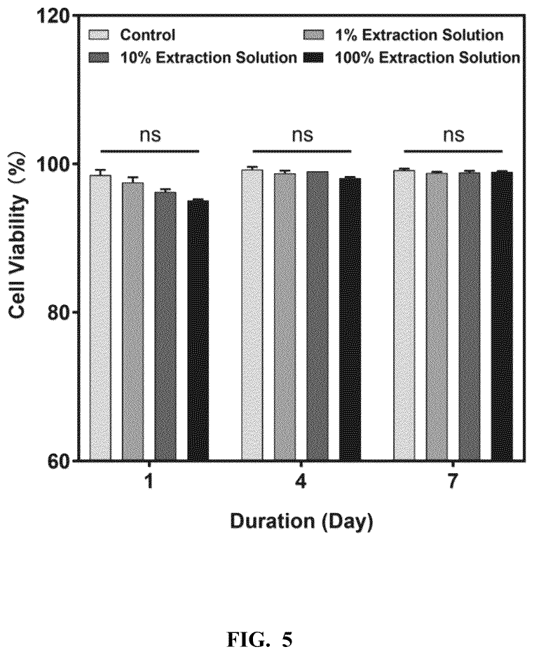

Live/dead staining: cells cultured for 1, 4, and 7 days were taken and stained using a cell double staining kit (Calcein AM/PI). The results are shown in FIG. 4 and FIG. 5, where 1%, 10%, and 100% in FIG. 5 refer to the proportion of the extraction solution. For example, 1% indicates 1% of extraction solution and 99% of culture medium.

As shown in FIG. 4 and FIG. 5, the porous hydrogel microspheres of Example 1 have good biocompatibility.

### Test Example 3

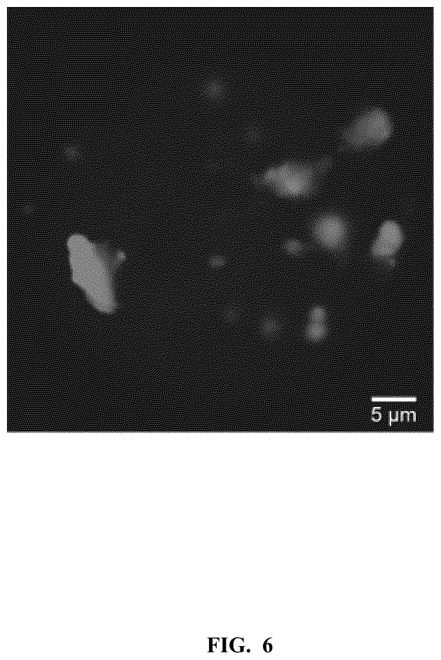

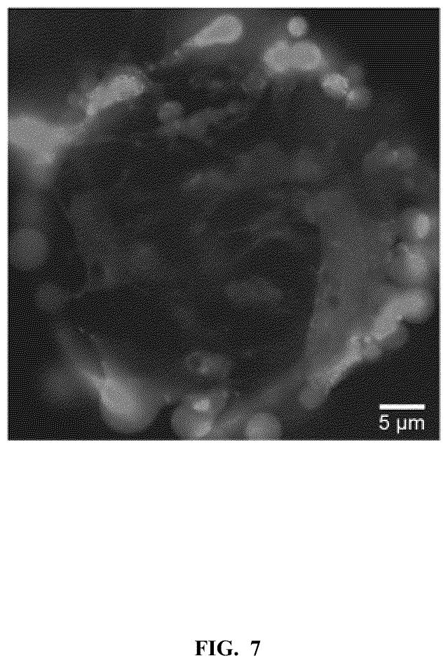

The porous hydrogel microspheres prepared in Example 1 were observed under a fluorescence microscope on day 1 and day 7. The results are shown in FIG. 6 and FIG. 7.

The results demonstrate that the cells have good expansion and proliferation within the porous hydrogel microspheres of Example 1.

### Test Example 4

The gelatin phase of the hydrogel and the pore-forming phase were prepared. The mass concentration of gelatin methacryloyl in the gelation phase was 0.1 g/mL, and the gelatin methacryloyl was dissolved in phosphate-buffered saline (PBS). The volume concentration of polyethylene glycol diacrylate in the gelation phase was 1%, and the mass concentration of lithium phenyl (2,4,6-trimethylbenzoyl) phosphinate (LAP photoinitiator) was 0.005 g/mL. The mass concentration of polyethylene oxide in the pore-forming phase was 0.016 g/mL, and the polyethylene oxide was dissolved in PBS. The mass concentration of gelatin in the pore-forming phase was 0.084 g/mL, and the polyethylene oxide was dissolved in PBS. The pore-forming phase was loaded with bone marrow mesenchymal stem at a concentration of 2x107 cells/mL.

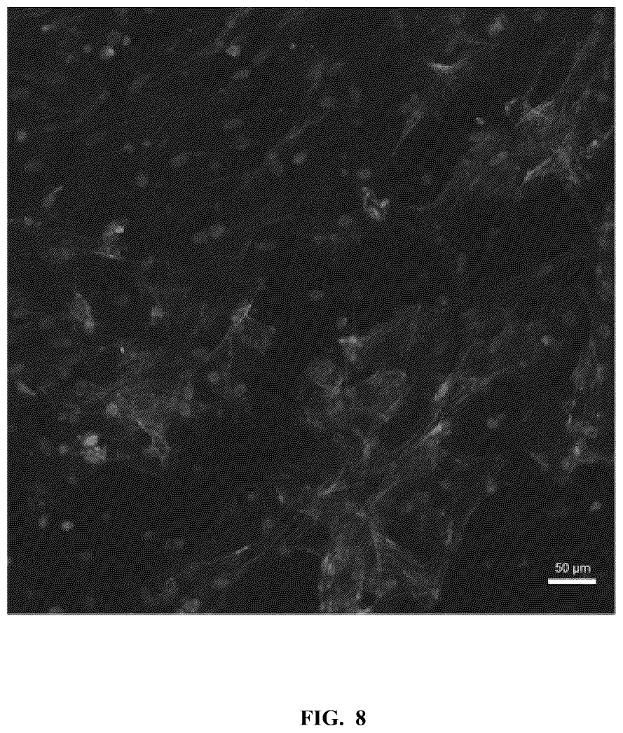

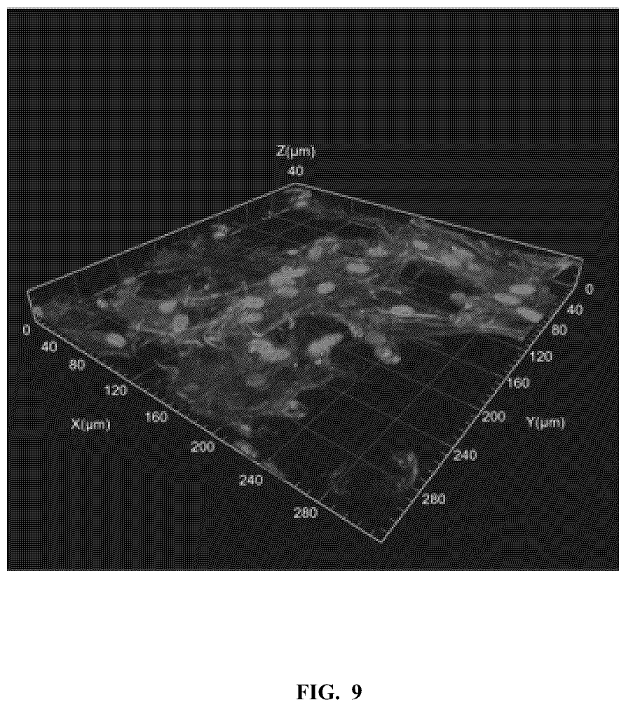

The gelation phase and the pore-forming phase were mixed and blowing for 8s, and ultraviolet crosslinking was performed for 15 s. After 7 days of cultivation, the cells were stained with phalloidin, and the expansion and proliferation of the bone marrow mesenchymal stem cells were observed using a confocal microscope. The results are shown in FIG. 8 and FIG. 9.

The results demonstrate that the cells have good expansion and proliferation in the porous hydrogel microspheres of Example 1.

### Test Example 5

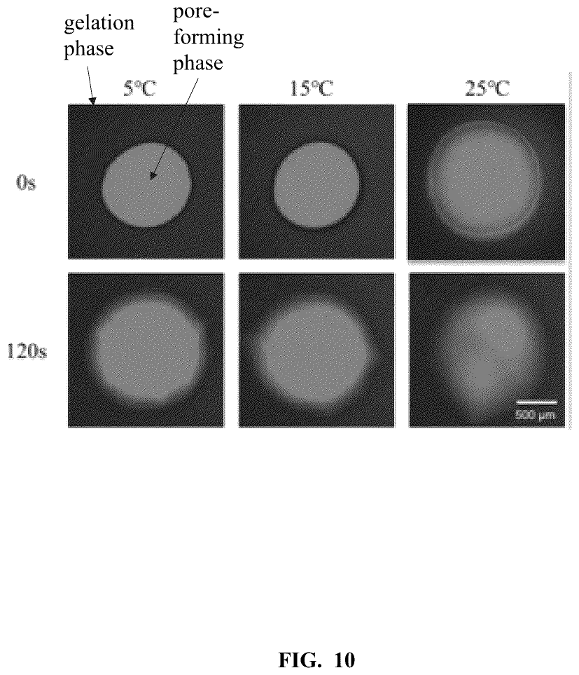

2 μL of the gelation phase of the hydrogel of Example 1 was added, and 1 μL of the pore-forming phase of Example 1 was then added dropwise to the gelation phase. The temperature of each group was controlled at 5° C., 15° C., and 25° C., respectively for 120 s during the addition process. The samples were then observed under a fluorescence microscope, and the results are shown in FIG. 10.

The results shown in FIG. 9 reveal that when the temperature is 25° C., the gelation phase of the hydrogel and the pore-forming phase were partially fused, however, when the temperature is 5° C. or 15° C., the gelation phase of the hydrogel and the pore-forming phase are in a stably separated state. It is indicated that the temperature is controlled at 5° C. or 15° C. in step (1), which is beneficial for obtaining porous hydrogel microspheres with stable morphology.

### Test Example 6

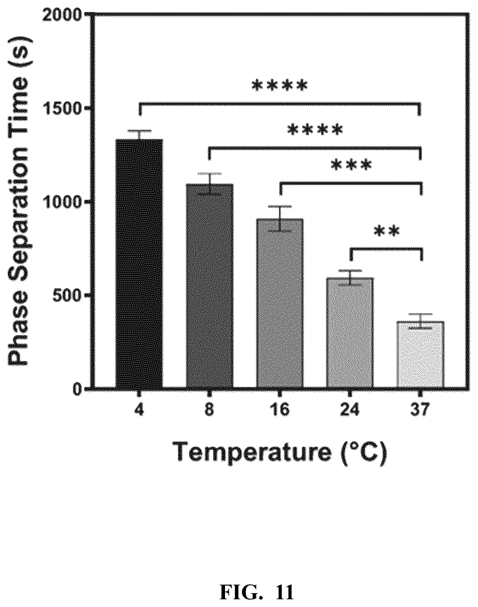

The pore-forming phase without loaded cell in Example 1 was treated at 4° C., 8° C., 16° C., 24° C., and 37° C., the phase separation time for the pore-forming phase was recorded, and the results were shown in FIG. 11. As shown in FIG. 11, it is observed that the lower the temperature, the longer the phase separation time of the pore-forming phase, the higher the temperature, the shorter the phase separation time of the pore-forming phase. It is indicated that the temperature is controlled at 5° C. and 15° C., which is beneficial for maintaining the pore-forming phase in a mixed state.

The basic concepts have been described above. Obviously, for those skilled in the art, the above detailed disclosure is only for example and does not constitute a limitation of the present disclosure. Although not explicitly stated here, those skilled in the art may make various modifications, improvements and corrections to the present disclosure. Such modifications, improvements and corrections are suggested in the present disclosure, so such modifications, improvements and corrections still belong to the spirit and scope of the exemplary embodiments of the present disclosure.

At the same time, the present disclosure uses specific words to describe the embodiments of the present disclosure. For example, “one embodiment”, “an embodiment”, and/or “some embodiments” refer to a certain feature, structure or characteristic related to at least one embodiment of the present disclosure. Therefore, it should be emphasized and noted that “one embodiment” or “an embodiment” or “an alternative embodiment” mentioned twice or more in different places in the present disclosure does not necessarily refer to the same embodiment. In addition, certain features, structures, or characteristics in one or more embodiments of the present disclosure may be appropriately combined.

Similarly, it should be noted that in order to simplify the description disclosed in the present disclosure and thus help understand one or more embodiments of the invention, in the above description of the embodiments of the present disclosure, a plurality of features are sometimes combined into one embodiment, figure, or description thereof. However, this disclosure method does not mean that the features required by the subject matter of the present disclosure are more than the features mentioned in the claims. In fact, the features of the embodiments are less than all the features of the single embodiment disclosed above.

In some embodiments, numbers describing the number of components and attributes are used. It should be understood that such numbers used in the description of the embodiments are modified by the modifiers “about”, “approximately” or “substantially” in some examples. Unless otherwise specified, “about”, “approximately” or “substantially” indicate that the numbers are allowed to vary by ±20%. Accordingly, in some embodiments, the numerical parameters used in the description and claims are approximate values, which may change according to the required features of individual embodiments. In some embodiments, the numerical parameters should take into account the specified significant digits and adopt the general method of retaining the digits. Although the numerical domains and parameters used to confirm the breadth of the range in some embodiments of the present disclosure are approximate values, in specific embodiments, the setting of such numerical values is as accurate as possible within the feasible range.

Each patent, patent application, patent application publication, and other materials, such as articles, books, specifications, publications, documents, etc., cited in the present disclosure is hereby incorporated by reference in its entirety. Except for application history documents that are inconsistent with or conflicting with the contents of the present disclosure, documents that limit the broadest scope of the claims of the present disclosure (currently or later attached to the present disclosure) are also excluded. It should be noted that if the descriptions, definitions, and/or use of terms in the materials attached to the present disclosure are inconsistent or conflicting with the contents described in the present disclosure, the descriptions, definitions, and/or use of terms in the present disclosure shall prevail.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be considered consistent with the teachings of the present disclosure. Accordingly, the embodiments of the present disclosure are not limited to the embodiments explicitly introduced and described in the present disclosure.

1. A method for preparing porous hydrogel microspheres, wherein the porous hydrogel microspheres are loaded with cells, and the method comprises:
mixing a gelation phase and a pore-forming phase at a temperature of 5-15° C. and forming first droplets through a shear force using a droplet microfluidic chip; wherein
the gelation phase comprises gelatin methacryloyl, polyethylene glycol diacrylate, and a photoinitiator; a concentration of gelatin methacryloyl in the gelation phase is within the range of 0.05-0.15 g/mL, a volume concentration of polyethylene glycol diacrylate is within the range of 0.5-3%, and a concentration of the photoinitiator is within the range of 0.001-0.005 g/mL; and
the pore-forming phase comprises polyethylene oxide and gelatin, a concentration of polyethylene oxide in the pore-forming phase is within the range of 0.01-0.016 g/mL, and a concentration of gelatin is within the range of 0.05-0.1 g/mL;
shearing a droplet stream formed by the first droplets through an oil phase to form second droplets;
curing the second droplets under an ultraviolet light to form hydrogel microspheres; and
removing the pore-forming phase from the hydrogel microspheres to obtain the porous hydrogel microspheres; wherein
the droplet microfluidic chip communicates with a processor and a temperature control component;
a structure of the droplet microfluidic chip includes a plurality of liquid injection holes, the plurality of liquid injection holes including:
a first liquid injection hole,
a second liquid injection hole,
a third liquid injection hole,
the structure of the droplet microfluidic chip further includes:
a first flow channel connected to the first liquid injection hole, a second flow channel connected to the second liquid injection hole, and a third flow channel connected to the third liquid injection hole,
the second flow channel being provided with a plurality of branch flow channels spaced apart from each other, each of the branch flow channels being arranged perpendicular to the first flow channel to form a T-shaped shear,
the third flow channel crossing the first flow channel in a cross shape, and
the gelation phase being introduced into the first flow channel from the first liquid injection hole, and the pore-forming phase being introduced into the second flow channel from the second liquid injection hole, the oil phase being introduced from the third liquid hole, and the gelation phase, the pore-forming phase, and the oil phase being introduced by an automatic liquid injection device.

2. The method for preparing the porous hydrogel microspheres according to claim 1, wherein the photoinitiator is lithium phenyl (2,4,6-trimethylbenzoyl) phosphinate.

3. The method for preparing the porous hydrogel microspheres according to claim 1, wherein the oil phase consists of dimethylsilicone oil and a surfactant, and the surfactant consists of one of decamethylcyclopentasiloxane and trimethylsiloxy silicate.

4. (canceled)

5. The method for preparing the porous microspheres according to claim 1, wherein the pore-forming phase is loaded with cells and after curing the second droplets under the ultraviolet light to form the hydrogel microspheres, the cells are adhered to pore structures of the hydrogel microspheres.

6. The method for preparing the porous hydrogel microspheres according to claim 5, wherein the cells consist of at least one of bone marrow mesenchymal stem cells, fibroblasts, and adipose-derived mesenchymal stem cells.

7. The method for preparing the porous hydrogel microspheres according to claim 6, wherein the oil phase consists of hydrofluoroether and nonionic fluorocarbon surfactant, a volume of the nonionic fluorocarbon surfactant being 1-2% of a volume of the hydrofluoroether, wherein the nonionic fluorocarbon surfactant is a single compound.

8-9. (canceled)

10. A porous hydrogel microspheres, which is prepared by the method of claim 1.
